(19) United States
(12) Patent Application Publication
(10) Pub. No.: US 2026/0041554 A1
(43) Pub. Date: Feb. 12, 2026

(54) SUTURE CONSTRUCTIONS FOR ADJUSTABLE TEMPORARY AND PERMANENT FIXATION OF A HEART VALVE LEAFLET RELATIVE TO A PAPILLARY MUSCLE DURING A CHORD REPAIR PROCEDURE

(71) Applicant: Nikola Dobrilovic, LLC, Chicago, IL (US)

(72) Inventor: Nikola Dobrilovic, Chicago, IL (US)

(73) Assignee: Nikola Dobrilovic, LLC, Chicago, IL (US)

(21) Appl. No.: 18/795,276

(22) Filed: Aug. 6, 2024

(51) Int. Cl. A61F 2/24

(52) U.S. Cl. CPC A61F 2/2457; A61F 2220/0008; A61F 2250/0004

(57) ABSTRACT

A suture construction for adjustable fixation of a heart valve leaflet relative to a papillary muscle or other supporting heart tissue is defined by a central body portion having a plurality of longitudinally spaced indexing openings or loops, an anchor structure at a first end of the body portion, and a suturing structure at a second end of the body portion. The anchor structure is secured or coupled to the papillary muscle or supporting heart tissue and the suturing structure is passed through the heart valve leaflet where one of the indexing loops is selectively fixed to the heart valve leaflet to adjustably fix the heart valve leaflet in a desired functional position relative to the supporting heart tissue. The indexing loops of the suture construction prevent unwanted movement of the heart valve leaflet in both directions and are securable in very small incremental positions for proper functional spacing.

### BACKGROUND OF THE DISCLOSURE

(1) Field of the Invention: The present disclosure relates generally to heart valve repair techniques and more particularly to the replacement or repair of heart valve chordae with sutures or other replacement chordae materials.

(2) Description of Related Art: Mitral valve regurgitation occurs when the tiny chords (chordae) that hold the mitral valve leaflets break or stretch and prevent the valve from sealing and closing properly. This causes your valve to leak (regurgitation). During a repair procedure, the mitral valve may be repaired by replacing the stretched or broken chords with lengths of sutures to create new chords (See FIGS. 1 and 2).

The basic procedure requires connecting (or reconnecting) the loose valve leaflet to the associated papillary muscle with a length of suture anchored at the papillary muscle and secured to the leaflet. A critical aspect of replacing the stretched or broken chordae is determining the proper length of the replacement chord (suture). This is particularly problematic as the original chord is damaged or stretched and thus it is difficult to ascertain its original length. Further, because the papillary muscle and leaflet tissues are soft and sutures tend to migrate or slide and may not retain their intended position.

Achieving the correct neochord length is simple in theory, but difficult in practice, as it must be performed in an incredibly precise manner with living pliable tissue. Success with this technique depends almost entirely on producing a neocord of the exactly correct length. The technique will likely fail if the neochord length is either slightly too long or slightly too short.

Even if the surgeon has a good estimate in mind, selecting the exact, final length is still difficult. Ideally, the surgeon would have the option of performing multiple, quick, temporary iterations by which the optimal length can be identified before permanently tying the suture knots down. Further, as noted above, tying the knots down without moving the fixation position can be quite difficult for a variety of reasons, including that the sutures simply slide, thus not retaining their intended position.

Many repair techniques using sutures involve tying successive overlapped knots along the length of the suture in an attempt to provide a defined fixed length. However, because of slippage, this technique is not usually successful and more importantly it is difficult to untie the knots to shorten the length when too long.

Various techniques have been employed by surgeons to limit or prevent suture migration in relation to the leaflet edge. Each of these techniques is deficient in one or multiple ways. One example is the placement of clips to secure the desired position. This method is sub-optimal because clips can damage the suture and can easily be lost into the patient (which could result in a stroke). Further, clips often fail in their intended purpose, as they do not secure the position well, and the knots can still slide during tying despite the presence of a clip. There is also a tradeoff between tighter/weaker clip placement. A stronger clip may decrease suture migration but may also weaken the suture more significantly.

Other techniques attempt to limit suture movement also fail for a variety of reasons. Examples include suturing the damaged leaflet to the normal leaflet while applying traction. While such methods are useful, immobilization is only partially, and not completely, achieved.

There are other techniques that attempt to aid in the identification and then construction of precise neochord length, but the techniques also remain inadequate. Other stabilization techniques include tying, using as reference an immobilized paired leaflet, or, tying against a guide such as the LivaNova ring. These methods attempt to provide a degree of immobilization but are prone to failure because they only provide partial immobilization, making them inadequate. For example, some of these techniques neglect to immobilize the papillary muscle (and/or similar tissue supporting structures). In such a scenario, though the surgeon is prevented from “pushing” the knot down too far/hard, there is nothing to prevent the surgeon from “pulling” the papillary muscle upward, suture and all. Such an unrestricted upward pulling motion allows for immediate, and often imperceptible, distortion of the neo-chord length, causing it to become shortened. As a result, many of the current methods provide a lesser or entirely inadequate degree of immobilization that can easily be overcome by unintentionally tying too forcefully. This is certainly true with the clip method, but also the others mentioned. Furthermore, iteration is much less precise and slow.

### SUMMARY OF THE DISCLOSURE

Accordingly, there is a need for a valve leaflet fixation device that is safe, reliable, durable, precise, reproducible, and quick to allow for iterations. Further, there is a need for a fixation device that facilitates the determination of precise neochord length in valve repair surgery by providing a bi-directionally stable, easily and continuously adjustable fixation device upon which to measure, test function and tie (secure) the neochord replacements.

The primary unresolved problem as discussed above relates to the inadequate fixation of the valve leaflet relative to the papillary muscle. In various embodiments of the disclosure, the invention provides for a suture construction that prevents (not just limits) movement of the leaflet being repaired in both directions, both towards and away from the surgeon, in a safe manner. Further the suture constructions provides for quick, precise adjustments to allow for easy iteration as this is a highly desired and essentially necessary feature of the neo-chord valve repair technique. The adjustability of the suture construction allows the surgeon to “dial-in” the optimal suture (replacement chord) length.

According to exemplary embodiments of the invention, a suture construction for adjustable temporary and/or permanent fixation of a heart valve leaflet relative to a papillary muscle or other heart tissue is defined by a central body portion having a plurality of longitudinally spaced indexing openings, a papillary/tissue anchor structure at a first end of the central body portion, and a leaflet suturing structure at a second end of the central body portion.

The suture construction may comprise a single suture strand looped back on itself to provide an anchor loop at one end with two parallel strands, where the strands are permanently or detachably fused at a plurality of longitudinally spaced locations to provide the indexing loops. Some embodiments may include an anchor loop at one end and a single tail suture needle at the opposing end. Other embodiments may have single or double tail suture strands with needles at one or both ends of the central body.

The suture structure may also comprise two parallel strands permanently or detachable fused together at a plurality of longitudinally spaced locations to provide the indexing loops. Some double strands embodiments may include a single tail suture needle at each end. Other embodiments may have single or double tail suture strands with needles at one or both ends of the central body.

In use, the anchoring structure (loop or suture tail) is secured or coupled to the papillary muscle or other supporting heart tissue and the leaflet suturing structure is passed through the heart valve leaflet where a predetermined one of the plurality of indexing openings is selectively fixed to the heart valve leaflet to selectively and adjustably fix the heart valve leaflet in a desired functional position relative to the papillary muscle or supporting tissue. The suture construction can be temporarily secured in place using one or more of the indexing loops to confirm proper chord length, and then permanently secured either with the integrated suture tails, or a separate suture stitch or stitches connecting the indexing loop(s) to the leaflet.

In further embodiments, the suture construction employs large suture loops to couple one or more of the above-described suture constructions between the papillary muscle and the leaflet.

The embodiments of the suture constructions described herein prevent unwanted movement of the heart valve leaflet in both directions and is adjustably securable in very small incremental positions for proper functional positioning.

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 is an illustration of a healthy heart valve anatomy with fully intact chords secured between the papillary muscle and the free edges of the heart valve leaflet;

FIG. 2 is an illustration of a damaged heart valve anatomy with ruptured chords detached from the free edges of the heart valve leaflet;

FIG. 3 is a plan view of an exemplary embodiment of suture construction for adjustable fixation of a heart valve leaflet relative to a papillary muscle in accordance with the teachings of the present disclosure;

FIG. 4 is an enlarged view showing the formation of indexing opening in the central body of the suture construction;

FIG. 4A is a view of an alternative arrangement including a spacing strand for the formation of indexing openings in the suture construction;

FIG. 5 is an illustration of the anchor loop of the suture construction being anchored to the papillary muscle;

FIG. 6 is a further illustration of the indexing loops passed through the leaflet edge and a single fixing suture secured through a selected indexing loop for temporary or permanent securing of the suture construction in place;

FIG. 7 is another illustration of the suture construction in use, wherein the indexing loops are passed through aligned slits in the leaflet edge and the terminal ends of the suture tails passed back through several of the loops for temporary or permanent securing of the suture construction in place;

FIG. 8 is yet another illustration showing use of two suture constructions with the indexing loops passed through the leaflet edge in adjacent relation and a first one of the suture constructions passed through a loop of the second one of the suture constructions for temporary or permanent securing of the suture constructions in place;

FIG. 9 is a plan view of another exemplary embodiment of suture construction for adjustable fixation of a heart valve leaflet relative to a papillary muscle in accordance with the teachings of the present disclosure;

FIG. 10 is an illustration of the anchor loop of the suture construction anchored to the papillary muscle and the indexing loops passed through the leaflet edge with the single suture tail and needle passed through a selected indexing loop for temporary or permanent securing of the suture construction in place;

FIG. 11 is a plan view of yet another exemplary embodiment of a suture construction for adjustable fixation with first and second suture tails at each end of the looped body portion;

FIG. 12 is a plan view of still another exemplary embodiment of a suture construction for adjustable fixation with single suture tails at each end of the looped body portion;

FIG. 13 is a plan view of another exemplary embodiment of a suture construction for adjustable fixation with two spaced sections of indexing loops and first and second suture tails at both end of the looped body portion;

FIG. 14 is an illustration of the suture construction of FIG. 13 in use with a center un-looped portion of the body anchored through the papillary muscle and the indexing loops extending upwardly to pass through the leaflet edge;

FIG. 15 a plan view of another exemplary embodiment of a suture construction for adjustable fixation with two spaced sections of indexing loops and single suture tails at each end of the body portion;

FIG. 16 is a plan view of an exemplary embodiment of a suture construction utilizing a single large loop suture to couple multiple looped suture constructions between the papillary muscle and the leaflet edge;

FIG. 17 is a plan view of another large loop embodiment of a suture construction utilizing a plurality of large loop sutures to couple multiple looped suture constructions between the papillary muscle and the leaflet edge;

FIG. 18 is a perspective view of an exemplary suture construction showing an enlargement of a fenestrated monofilament central body portion providing the indexing loops;

FIG. 19 is an enlarged perspective view of another exemplary fenestrated monofilament suture construction showing multiple repeating sets of fenestrated openings in the central body portion providing the indexing loops; and

FIG. 20 is an enlarged perspective view of an exemplary suture construction showing braided multi-filament suture central body portion providing indexing locations within the central body portion.

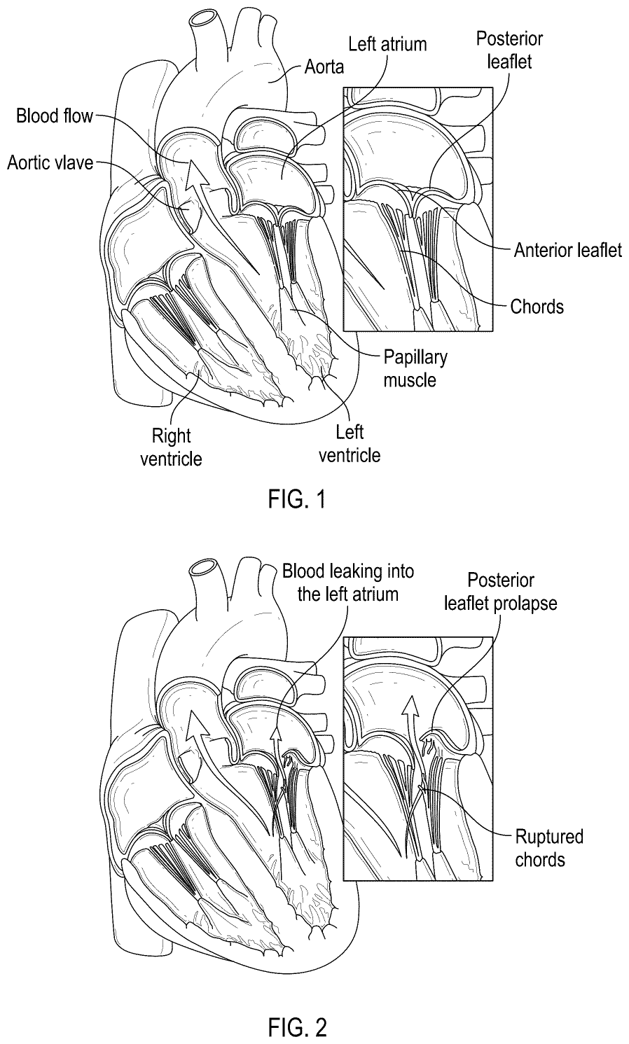

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

In various embodiments of the disclosure, the invention provides for a suture construction that prevents (not just limits) movement of the leaflet being repaired in both directions, both towards and away from the surgeon, in a safe manner. Further, the suture constructions provides for quick, precise adjustments to allow for easy iteration as this is a highly desired and essentially necessary feature of the neo-chord valve repair technique. The adjustability of the suture construction allows the surgeon to “dial-in” the optimal suture (replacement chord) length.

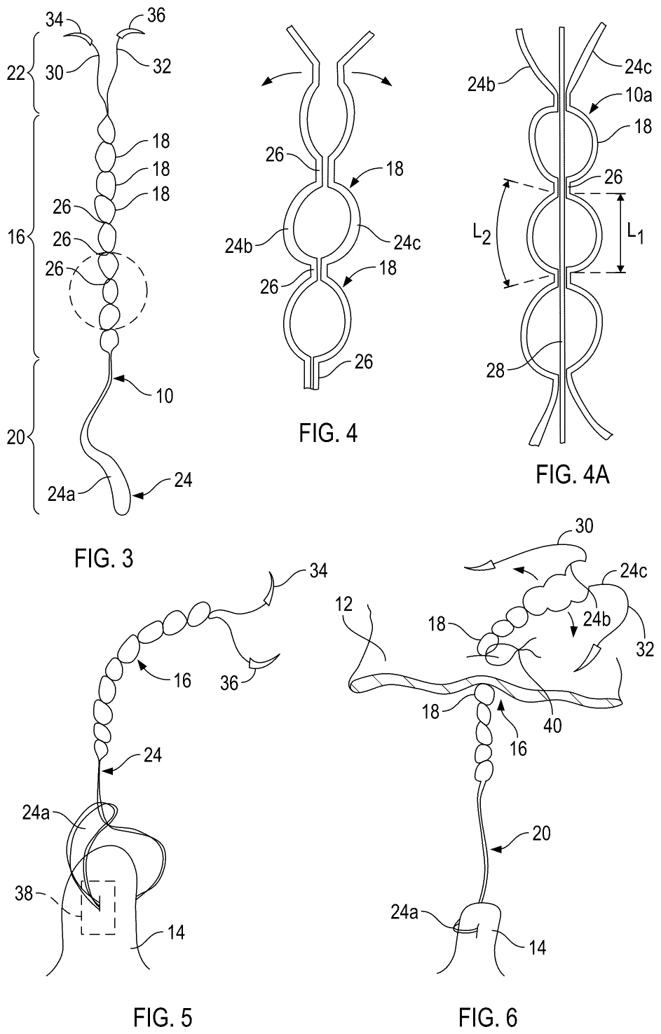

According to exemplary embodiments of the invention, and referring now to FIGS. 3-7, an exemplary suture construction 10 for adjustable temporary and/or permanent fixation of a heart valve leaflet 12 relative to a papillary muscle 14 (or other suitable supporting heart tissue) is defined by a central body portion 16 having a plurality of longitudinally spaced indexing openings or loops 18, a papillary anchor structure 20 at a first end of the central body portion 16, and a leaflet suturing structure 22 at a second end of the central body portion 16.

Referring to FIGS. 3 and 4, a suture construction 10 may comprise a single suture strand 24 looped back on itself to provide an anchor loop 24A at one end with two parallel strands 24B, 24C (FIG. 4) where the strands 24B, 24C are permanently or detachably fused or secured at a plurality of longitudinally spaced locations 26 to provide a plurality of longitudinally spaced indexing loops 18. As best seen in FIG. 4, the suture strand 24 may be naturally or artificially pre-biased or shaped into the mating parts and curved loop shapes as illustrated in order to facilitate fusing, adhering or other securement.

The suture strand(s) 24 may comprise non-bio-absorbable synthetic suture materials such as nylon, polyester and other suitable polymer materials which can be permanently or detachably fused by various manufacturing methods. In some embodiments, the suture material may comprise polytetrafluoroethylene (PTFE).

Contemplated manufacturing techniques for creating the open loops and permanent or detachable bonds at select locations between the strands include heat welding, sonic welding, mechanical staking, adhesives and crimped fasteners. The term “loops” may be broadly interpreted to include circular or oval loops or other suitable shapes with an open interior space capable of receiving a securing suture therethrough.

The suture strand 24 may be monofilament, multi-filament, pseudo-multifilament, twisted filament or braided filament constructions as known the in suture arts.

In some embodiments, portions of the sutures, or selected spaced loops, may be colored to identify various locations or distances along the suture length. Varying colors may also be used to easily identify length or position.

Referring briefly to FIG. 4A, due to the elastic memory of many polymer materials, the open loops in polymer suture materials which are created in the manufacturing process may elongate naturally or under tension (pulled from end to end) and may tend to close up after formation, thereby elongating the length of the suture construction (a known fixed length is critical).

Further, it is obviously preferred that the looped openings 18 remain completely open to facilitate visibility, manipulation and final fixation to the tissue structures. In this regard, an exemplary suture construction embodiment 10A, as illustrated in FIG. 4A, and depending on polymer suture material, may further comprise a linear spacing strand 28 positioned and fused or secured between the parallel suture strands 24B, 24C to maintain the loops 18 open. In this regard, a length of the spacer strand 28 (L1) within the loop 18 will be fixed and secured in conjunction with longer curved lengths (L2) of the suture strands 24B, 24C to maintain an open condition of the loop 18, even under tension.

Natural suture materials such as silk are also contemplated, however other means for creating permanent or detachable loops will need to be considered, such as crimped surgical metal fasteners, tied knots or the like.

Turning back to FIGS. 3, 5, 6 and 7, the exemplary suture construction 10, the leaflet suturing end 22 may be provided with first and second suturing tails 30, 32, each having a respective suture needle 34, 36 crimped or otherwise attached to the ends thereof.

Needle shapes may vary from ¼ circle to ⅝ circle or otherwise as known in the art.

In some techniques, the anchoring structure 20 (anchor loop 24A) may be secured or coupled to the papillary muscle 14 by passing the suturing tails 30, 32 through the papillary muscle 14 and then looping the tails 30, 32 through the loop end 24A (FIG. 5). In other techniques, the anchoring structure 20 may be passed through another suitable supporting heart tissue near or adjacent to the papillary muscle 14 as determined by the attending surgeon.

A pledget 38 (shown in broken line) (or multiple pledgets) may be utilized to provide stability. The leaflet suturing tails 30, 32 may then be passed through a free edge of the heart valve leaflet 12, or other suitable location in the leaflet, where a predetermined one of the plurality of indexing openings 18 is selectively fixed to the heart valve leaflet 12 to selectively and adjustably fix the heart valve leaflet 12 in a desired functional position relative to the papillary muscle 14 (FIG. 6). The suture construction 10 can be temporarily secured in place using one or more of the indexing loops 18 to confirm proper chord length (or another temporary suture stitch), and then permanently secured either with the integrated suture tails 30, 32, or a separate suture stitch 40 or stitches connecting the indexing loop(s) 18 to the leaflet 12 (FIG. 6).

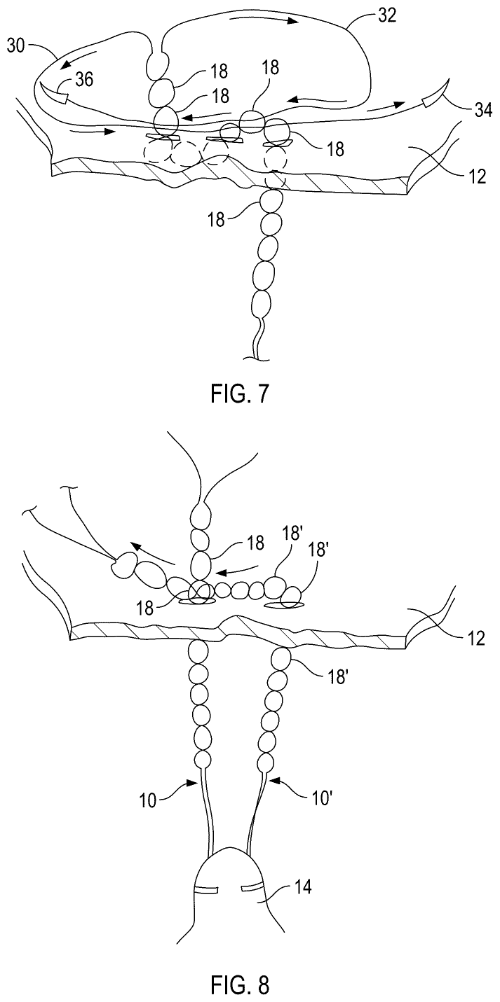

As seen in FIG. 7, the indexing loops 18 may be passed through the leaflet edge multiple time to provide a stable fixing point and then the suture tails 30, 32 passed back through one or more of the loops 18 to temporarily or permanently fix the suture construction 10 in place.

As also seen in FIGS. 4, 6 and 7, where the indexing loops 10 are formed with a detachable fused bond or are otherwise made to be selectively separable, the suture strands 24B, 24C can be separated to a point adjacent the leaflet 12 and then used to permanently tie the suture construction 10 in place to form a permanent replacement chord.

When the integrated suture tails 30, 32 are used to permanently fix the suture construction 10 in place, the suture construction become a fully integrated “self-contained” adjustable fixation solution.

Turing to FIG. 8, another repair technique is illustrated using two side-by-side suture constructions 10, 10′, each anchored to the papillary muscle 14, and both passing through the leaflet edge 12. In this repair scenario, the second of the suture constructions 10′ is passed through a selected loop 18 (or loops) on the first of the suture constructions 10 to provide both temporary adjustable fixation and thereafter permanent suture fixation to provide a stable multi-position chord replacement.

Additional embodiments will be described herein where each embodiment includes a corresponding central body portion with an anchoring end and a leaflet suturing end as described hereinabove, even though each may not be specifically numbered or labeled, the respective descriptions hereinabove are equally applicable to respective corresponding elements.

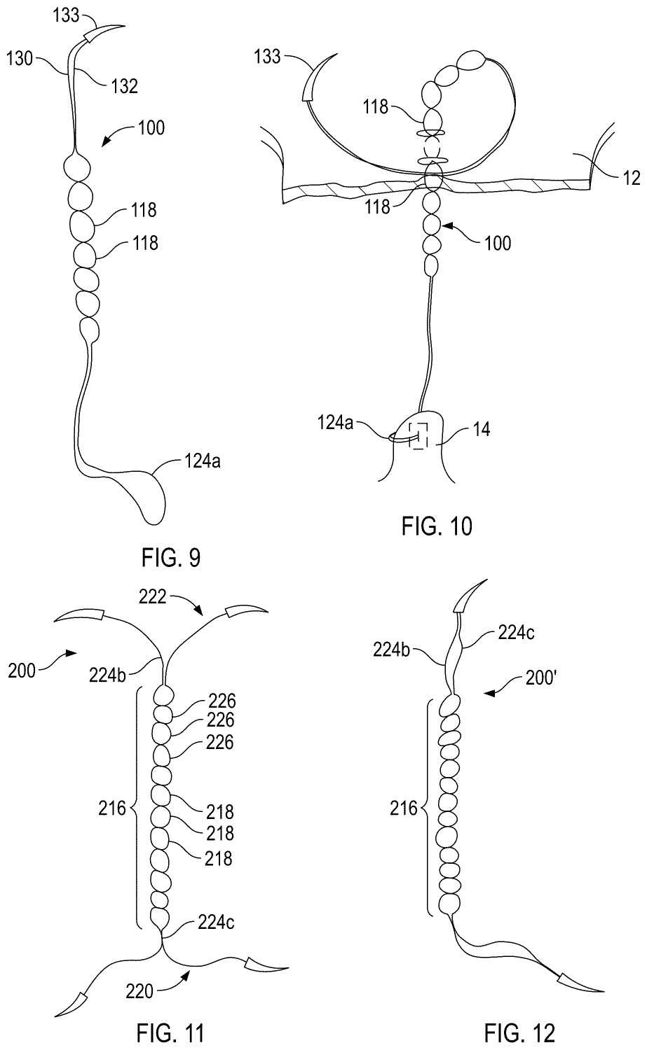

Another single strand embodiment 100 may include a central body with indexing loops 118, an anchor loop 124A at one end and a single tail suture needle 133 at the opposing end (FIG. 9). The suture tails 130, 132 are joined and crimped together with the needle 133. As shown in FIG. 10, an exemplary repair technique passes the indexing loops 118 through the leaflet edge and then loops the single suture tail and needle 133 back through one or more selected loops 118 to temporarily, and then permanently secure the suture construction 100 in place.

Referring now to FIG. 11, another exemplary suture construction 200 may comprise two separate parallel, side-by-side or adjacent suture strands 224B, 224C which are permanently or detachable fused or secured together at a plurality of longitudinally spaced locations 226 to provide a plurality of longitudinally spaced indexing loops 218 in the central body portion 216. The suture construction 200 includes an anchoring end 220 with first and second suture tails and respective suture needles as well as a leaflet suturing end 222 with first and second suture tails and respective suture needles.

FIG. 12 illustrates an alternative double strand suture construction 200′ where the first and second suture tails at both ends of the central body 216 are crimped into a single suture needle.

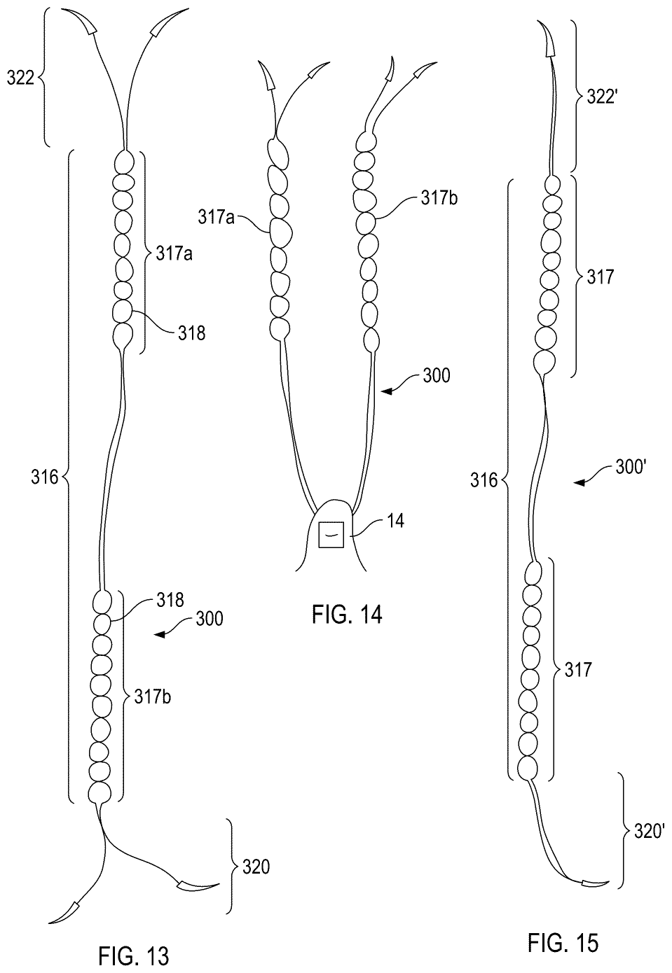

FIG. 13 illustrates another exemplary embodiment of a dual strand suture construction 300 for adjustable fixation comprising two spaced sections 317A, 317B of indexing loops 318 within the central body portion 316 and first and second suture tails at both the anchoring end 322 and the leaflet suturing end 322 of the body portion 316. FIG. 14 is an illustration of the suture construction 300 of FIG. 13 in use with a central un-looped portion of the central body 316 anchored through the papillary muscle 14 (with a pledget) and the indexing loop sections 317 extending upwardly to pass through a leaflet edge (not shown).

FIG. 15 illustrates an alternative suture construction 300′ for adjustable fixation with two spaced sections 317A, 317B of indexing loops 318 within the central body 316 and single suture tails and needles at both the anchoring end 322′ and the leaflet suturing end 322′ of the body portion 316.

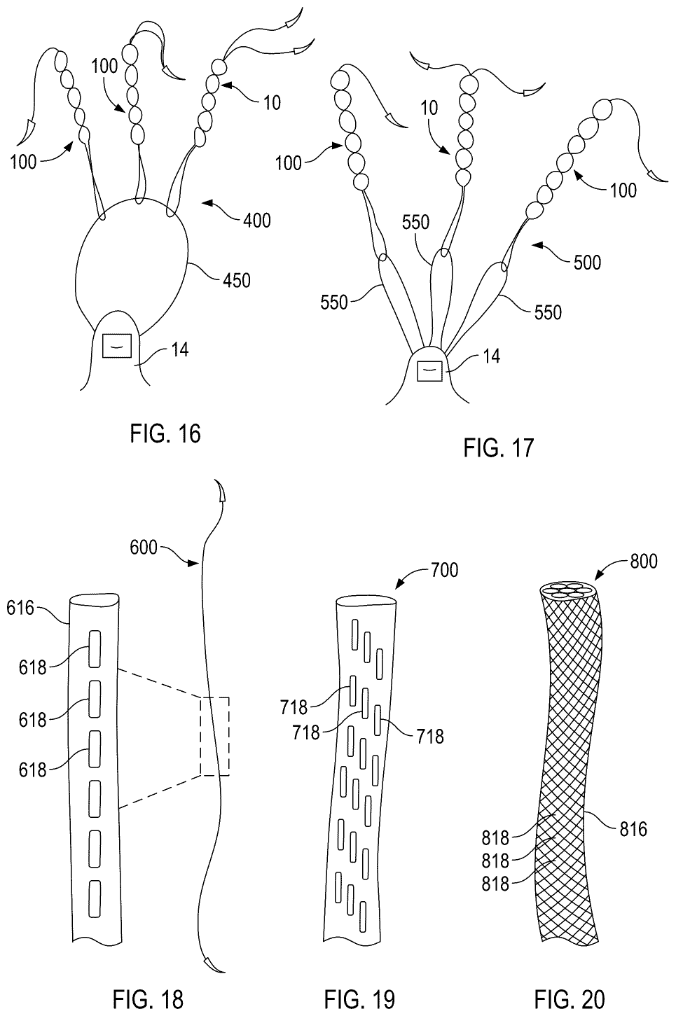

FIG. 16 illustrates still another exemplary embodiment of a suture construction 400 utilizing a single large loop suture 450 secured to the papillary muscle 14 to couple multiple looped suture constructions 10, 100 between the papillary muscle 14 and the leaflet edge (not shown).

FIG. 17 illustrates a further large loop embodiment of a suture construction 500 utilizing a plurality of large suture loops 550, anchored at a single location, to the papillary muscle 14 to couple multiple looped suture constructions 10, 100 between the papillary muscle 14 and the leaflet edge.

FIG. 18 is a perspective view of an exemplary suture construction 600 showing an enlargement of a fenestrated monofilament suture strand utilized as the central body portion 616 with the fenestrations 618 providing a plurality of longitudinally spaced indexing openings.

FIG. 19 is an enlarged perspective view of another exemplary fenestrated monofilament suture construction 700 showing multiple repeating sets of fenestrated openings 718 in the central body portion 716 providing the plurality of longitudinally spaced indexing loops.

FIG. 20 is an enlarged perspective view of an exemplary suture construction 800 showing a braided multi-filament suture central body portion 816 wherein the spaces 818 between the adjacent filaments provide an infinite number of indexing/securing locations within the central body portion 816 by allowing a suture tail or other independent suture to be threaded through the braided suture body 816.

In some embodiments, a single strand of the braided body portion 816 may be detachable from the body portion.

It can therefore be appreciated that the embodiments of the suture constructions described herein facilitate suture placement and proper leaflet fixation while preventing unwanted movement of the heart valve leaflet in both directions and being adjustably securable in very small incremental positions for proper functional positioning.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A suture construction for adjustable fixation of a heart valve leaflet relative to a supporting heart tissue comprising:
a central body portion having a plurality of longitudinally spaced indexing openings;
an anchor structure at a first end of the central body portion; and
a leaflet suturing structure at a second end of the central body portion,
wherein said anchor structure is secured or coupled to said supporting heart tissue, and
wherein said leaflet suturing structure is attached to the heart valve leaflet and a predetermined one of said plurality of indexing openings is selectively fixed to said heart valve leaflet to selectively and adjustably fix said heart valve leaflet in a desired functional position relative to said supporting heart tissue.

2. The suture construction of claim 1 wherein said plurality of longitudinally spaced indexing openings comprise a plurality of circular loops formed by parallel first and second suture strands secured together at longitudinally spaced intervals along the central body portion.

3. The suture construction of claim 2 wherein said parallel first and second suture strands are permanently secured at said spaced intervals.

4. The suture construction of claim 2 wherein said parallel first and second suture strands are detachably secured at said spaced intervals.

5. The suture construction of claim 1 wherein said anchor structure comprises a closed loop.

6. The suture construction of claim 1 wherein said anchor structure comprises an anchor suturing structure.

7. The suture construction of claim 1 wherein said suturing structure comprises a suture tail and with a suture needle.

8. The suture construction of claim 1 wherein said suturing structure comprises first and second suture tails with respective suture needles.

9. The suture construction of claim 1 further comprising a large loop suture secured between said anchor structure and said supporting heart tissue.

10. The suture construction of claim 1, further comprising:
a loop spacing strand extending longitudinally between the parallel first and second suture strands and selectively secured at said plurality of longitudinally spaced locations to maintain said indexing openings in an open position.

11. A suture construction comprising,
a single suture strand looped back on itself so that a first strand portion and a second strand portion thereof lie alongside each other in parallel relation forming an anchor loop at one end and first and second suture tails at the opposing end,
wherein the first and second strand portions are secured together at a plurality of longitudinally spaced locations to form a plurality of longitudinally spaced indexing loops along the length thereof.

12. The suture construction of claim 11, wherein the first and second strand portions are secured together permanently at said plurality of longitudinally spaced locations.

13. The suture construction of claim 11, wherein the first and second strand portions are detachably secured together at said plurality of longitudinally spaced locations.

14. The suture construction of claim 11, further comprising:
a loop spacing strand extending longitudinally between the first and second strand portions and selectively secured at said plurality of longitudinally spaced locations to maintain said indexing loops in an open position.

15. The suture construction of claim 11 wherein said first and second suture tails are joined with a suture needle.

16. The suture construction of claim 11 wherein said first and second suture tails have respective suture needles.

17. A suture construction comprising;
first suture strand; and
a second suture strand,
wherein the first and second suture strands lie alongside each other in parallel relation, and
wherein the first suture and the second suture are secured together at a plurality of longitudinally spaced locations to form a plurality of longitudinally spaced openings along the length thereof.

18. The suture construction of claim 17, wherein the first suture and the second suture are secured together permanently at said plurality of longitudinally spaced locations.

19. The suture construction of claim 17, wherein the first suture and the second suture are detachably secured together at said plurality of longitudinally spaced locations.

20. The suture construction of claim 17, further comprising:
a loop spacing strand extending longitudinally between the first suture and the second suture and selectively secured at said plurality of longitudinally spaced locations to maintain said indexing loops in an open position.
